(19) United States
(12) Patent Application Publication
(10) Pub. No.: US 2010/0278902 A1
(43) Pub. Date: Nov. 4, 2010

(54) STRONTIUM DOPED BIOACTIVE GLASSES

(76) Inventors: Edouard Jallot, Saint-Beauzire (FR); Jonathan Lao, Clermont-Ferrand (FR); Jean-Marie Nedelec, Moissat (FR); Jean-Michel Sautier, Paris (FR); Juliane Isaac, Paris (FR)

Correspondence Address: MORGAN LEWIS & BOCKIUS LLP, 1111 PENNSYLVANIA AVENUE NW, WASHINGTON, DC, 20004, US

(21) Appl. No.: 12/668,378

(22) PCT Filed: Jul. 8, 2008
(86) PCT No.: PCT/FR08/00985

§ 371 Date: Jul. 9, 2010

(30) Foreign Application Priority Data
Jul. 9, 2007 (FR) 0704952

(51) Int. Cl. A61K 9/70; A61F 2/28; A61K 33/42; A61P 19/00

(52) U.S. Cl. 424/443; 623/23.51; 424/605

(57) ABSTRACT

The invention relates to bioactive glasses containing or doped with strontium, to a method for preparing the same and to the use thereof in methods for bone repair or reconstruction.

The invention relates to novel bioactive glasses comprising or doped with strontium, a method for preparation thereof and use thereof in methods of bone repair or reconstruction.

The bones are constituted of a network of collagen fibers and hydrated and carbonated crystals of calcium phosphate. Cells called osteocytes, which comprise osteoblasts and osteoclasts, are inserted in this network. They are supplied by very small blood vessels.

When a bone is damaged, the osteoclasts remove the damaged fragments and the osteoblasts reconstruct the collagen network and promote the production of enzymes that will enable crystalline hydroxycarbonate apatite to be deposited, until the bone defect is repaired.

As this natural process is slow, it is usual to assist bone repair by means of bone cements or prostheses of varying size depending on the dimensions of the damaged region. A bone graft is sometimes necessary when reconstruction of the bone does not take place or is too slow.

In all cases of repair of a bone defect, it is important, in parallel with the placement of a replacement structure, to promote reconstruction of the bone tissue, which will progressively colonize or take the place of the bone substitute.

In certain diseases, and notably osteoporosis, it is important to counter the degradation of the bone tissue by stimulating the activity of the osteoblasts.

For all these applications, bioactive glasses have been under development for many years. Bioactive glasses react chemically with biological fluids, and the product of the reaction is a hydroxyapatite, which promotes formation of the bone matrix and bone growth.

The first bioactive ceramics were developed by L. L. Hench (L. L. Hench et al., J. Biomed. Mater. Res. 1971, 2, 117-141; L. L. Hench et al., J. Biomed. Mater. Res. 1973, 7, 25-42).

The first bioactive glasses were prepared from SiO₂ and P₂O₅ and from CaO and Na₂O. Oxides of silicon and of phosphorus are network formers, which participate in cohesion of the vitreous network. The alkali metals and alkaline-earth metals such as sodium and calcium do not have this capacity and will modify the vitreous network by introducing chain breaks into it, which are the cause of the low melting point of these glasses associated with increased structural disorder. Their presence leads to greater reactivity of bioactive glasses in an aqueous environment. This reactivity permits the formation of hydroxyapatite in a physiological environment and therefore promotes bone reconstruction.

The bioglass that has received most study is a sodium-silicon-phosphorus-calcium glass called Bioglass® or Bioverre by Hench. Its basic composition is 55% SiO₂-20% CaO-20% Na₂O-5% P₂O₅. The remarkable bioactive properties of this material require no further demonstration. Bioglass® is still one of the most interesting of the bioactive materials (inducing a specific response of the cells).

Many advances have been made in the field of bioactive glasses since their discovery (M. Vallet-Regi et al., Eur. J. Inorg. Chem. 2003, 1029-1042), such as the incorporation of various atoms or the incorporation of active principles. The compositions of the bioactive glasses have been optimized so as to promote the proliferation of osteoblasts and the formation of bone tissues (WO 02/04606). The incorporation of silver has been proposed notably for endowing bioactive glasses with antibacterial properties (WO 00/76486).

However, the incorporation of a new element in a bioactive glass always presents difficulties: in fact, any atom introduced in a composition of bioactive glass has an influence on the behavior of said glass and on its properties, in particular on the way in which this glass salts-out the elements of which it is composed. Moreover, the bioactive glass must also dissolve well to permit the formation of hydroxyapatite, but the rate of dissolution must be controlled to permit progressive colonization of the hydroxyapatite and prolonged salting out of any active substances.

Finally, the conditions of production of bioactive glasses must be adapted to each new composition.

The bioactivity properties of the glasses and their rate of dissolution depend on their composition and their texture. The basic composition of a bioactive glass is of the form SiO₂—CaO—P₂O₅ or SiO₂—Na₂O—CaO—P₂O₅. However, there has been very little study of the role of certain trace elements during the various stages in the process of dissolution, of salting out of ions and the physicochemical reactions leading to bioactivity.

Strontium is naturally present in bone tissues and it can be incorporated in the apatites during the phases of growth of the precipitates (formation of calcium-deficient apatites). Moreover, the literature describes this element as being able to exert an influence on cellular reactions. Strontium improves the mechanical properties of bone and it has an influence on the solubility of the apatites. It is also involved in osteoporosis since it improves the mechanical properties of the hydroxyapatites. It provides, in vivo, a better bond with the surrounding tissues. Although it improves cell adhesion, it slightly reduces growth of osteoblasts in culture and increases the production of lactate dehydrogenase. Strontium also makes it possible to immobilize cells, and the adhesion of cells might be better when the biomaterial is doped with Sr (E. Canalis et al., Bone 1996, 18, 517-523; G. Boivin et al., J. Bone Miner. Res. 1996, 11, 1302-1311; P. Marie and M. Hott, Metabolism 1986, 35, 547-551; P. Marie, Current Opinion in Pharmacology 2005, 5, 633-636).

One object of the invention was to develop a novel bioactive material that has improved properties relative to the materials of the prior art.

The material of the invention is a composition of bioactive glass comprising strontium. According to a first embodiment of the invention, this bioactive glass results from a sol-gel process. This composition is characterized by the presence of the following constituents in the proportions stated:

SiO₂: from 40 to 75%
CaO: from 15 to 30%
SrO: from 0.1 to 10%
P₂O₅: from 0 to 10%
Na₂O: from 0 to 20%
MgO: from 0 to 10%
ZnO: from 0 to 10%
CaF₂: from 0 to 5%
B₂O₃: from 0 to 10%
Ag₂O: from 0 to 10%
Al₂O₂: from 0 to 3%
MnO: from 0 to 10%
Others: from 0 to 10%

The percentages are percentages by weight relative to the total weight of the composition.

Advantageously, the sum of the weights of the constituents SiO₂, CaO, SrO, P₂O₅ represents 98 to 100%, better still 99 to 100%, and preferably 99.9 to 100% of the total weight of the composition of the material of the invention.

Advantageously, the material of the invention is constituted of:

SiO₂: from 45 to 75%

CaO: from 15 to 30%

SrO: from 2 to 8%

P₂O₅: from 0 to 10%

Other elements: from 0 to 1%, preferably from 0 to 0.5%, by weight relative to the total weight of the composition.

The materials of the invention can result from a sol-gel process and can be in the form of a loose powder or a compacted powder, in the form of fibers or alternatively in the form of a coating on a substrate or of a monolith or of a glass frit.

According to one embodiment of the invention, the materials can result from a high-temperature fusion process followed by quenching. In this case they are defined by the following composition:

SiO₂: from 45 to 55%

Na₂O: from 10 to 25%

CaO: from 10 to 25%

SrO: from 0.1 to 10%

P₂O₅: from 0 to 10%

MgO: from 0 to 10%

ZnO: from 0 to 10%

CaF₂: from 0 to 5%

B₂O₃: from 0 to 10%

Ag₂O: from 0 to 10%

Al₂O₂: from 0 to 3%

MnO: from 0 to 10%

Others: from 0 to 10%.

The percentages are percentages by weight relative to the total weight of the material.

Advantageously, the sum of the weights of the constituents SiO₂, Na₂O, CaO, SrO, P₂O₅ represents 98 to 100%, better still 99 to 100%, and preferably 99.9 to 100% of the total weight of the composition of the materials of the invention.

Advantageously, according to this embodiment, the material of the invention is constituted of:
SiO₂: from 45 to 55%
Na₂O: from 15 to 25%
CaO: from 15 to 25%
SrO: from 2 to 8%
P₂O₅: from 0 to 10%
Other elements: from 0 to 1%, preferably from 0 to 0.5%, by weight relative to the total weight of the composition.

The materials of the invention can result from a fusion process and can be in monolithic form or in the form of glass frit.

The expression “bioactive glass” denotes a material of the inorganic glass type in which silicon oxide is the main component, and which is capable of binding to living tissues when it is placed in a physiological fluid.

Bioactive glasses are well known by a person skilled in the art and are described notably in “An introduction to Bioceramics”, L. Hench and J. Wilson, World Scientific Edition, New Jersey (1993).

The materials of the invention are biocompatible, which means that when they are put in contact with a living organism, and notably with a human or animal organism, they do not induce a reaction of the organism's defense systems, such as the immune system in particular. The term biocompatible also implies that when the material is implanted in a patient, it does not produce cytotoxic effects or systemic reactions.

The materials of the invention are both biocompatible and bioactive. Relative to the materials of the prior art, they possess the advantage of reinforcing the mechanical properties of bone and of promoting bonding between hydroxyapatite and the surrounding tissues. The biomaterials of the invention therefore have properties that make them superior to the biomaterials of the prior art in the repair of bone defects and in the prevention and/or treatment of bone deficiencies of any origin.

The materials of the invention can be prepared by a sol-gel process, which offers many advantages: lower production temperatures than for other methods, materials that are more homogeneous, easy control of the final composition and control of the porosity and specific surface area of the material. As bioactivity is determined by the structure of the material as well as its chemical composition, it was found that the materials resulting from a sol-gel process were particularly interesting as it is easy, in this process, to control their rate of dissolution as well as the rate of salting out of strontium.

The materials of the invention can be prepared by a method that comprises the stages of mixing of the metal alkoxides in solution, hydrolysis, gel formation and heating so as to produce a porous matrix or a dense glass.

The sol-gel process is applied with a composition of material as described above with 3 components or more, including at least SiO₂, CaO, SrO, and optionally P₂O₅ and/or other oxides.

In a first stage the precursors of the components, the solvent (water and optionally an alcohol such as ethanol) are mixed in the presence of an acid or basic catalyst.

In more detail, a tetraalkoxysilane such as tetraethoxysilane is used as SiO₂ precursor, a trialkyl phosphate such as triethyl phosphate is used as P₂O₅ precursor, calcium nitrate tetrahydrate or another calcium salt (chloride, acetate, fluoride, oxalate etc.) is used as CaO precursor and strontium nitrate or another salt of strontium (chloride, acetate, fluoride, oxalate etc.) as SrO precursor.

The reactions of hydrolysis and condensation are catalyzed by the same catalyst, for example HCl. The structure of the gel that forms is notably determined by the pH of the solution in which these reactions take place. At the percolation threshold the three-dimensional network formed extends throughout the reaction mixture and a gel is obtained.

Aging: this stage involves keeping the gel immersed in the solvent for several hours to several days. During aging, polycondensations take place until all the reactive species have reacted. This stage, called syneresis, contributes to reduction of porosity and reinforcement of the gel. The porosity of the gel can be controlled by adjusting the duration and temperature of aging.

During the drying stage, the liquid present in the pores is expelled from them. Capillary stresses develop and cause the gel to crack, unless conditions are employed that reduce the solid-liquid interfacial strains, for example by adding surfactants.

Stabilization and densification can be obtained by thermal or chemical means, in conditions that make it possible to eliminate the silanol surface groups.

Preferably heating is employed, so as to degrade the other components present in the gel, such as the nitrates. Heating is preferably carried out at a temperature greater than or equal to 600° C.

The final product is then obtained in the form of a powder. The size of the pores is between 1 nm and 50 μm. Preferably, a powder is obtained with pores from 2 to 50 nm in diameter.

The uses to which the materials of the invention can be put are as follows: filling of bone defects, covering of metallic implants, stimulation of bone growth in cases of osseous degeneration.

These applications can be implemented in various ways:

The materials of the invention can be introduced locally by surgery or by injection: in a region where a bone defect has been found, for example by radiography. It is possible to fill a bone defect by inserting a material of the invention in the form of loose powder or compacted powder.

The powder obtained by the method can be used as it is, for example in bone surgery or maxillodental surgery, for filling bone defects. It can be injected in the form of a therapeutic composition in regions where stimulation of bone growth is required. This powder can be compacted in the form of tablets by means of a press, so as to form a three-dimensional object, which is used in surgery.

According to one embodiment of the invention, fibers of bioactive glass can be prepared by means of a sol-gel process, employing the following stages: the sol is extruded through a die. The fibers obtained are aged, dried and stabilized thermally. The fibers can then be woven or agglomerated by means of a binder, for example a solution of carboxymethylcellulose. A network of agglomerated fibers can then be used for producing a glass frit by heating in a stove at temperatures causing degradation of the binder.

The bioglass fibers of the invention can be used as they are, as suture thread or in the form of cloth, in surgery. They can be used in compositions that include other materials.

The materials of the invention can be used alone or in combination with other means promoting the repair and/or regeneration of bone tissue. Therapeutic compositions, notably compositions intended for injection or for administration by surgery, and comprising at least one material of the invention, constitute another object of the invention. These compositions can comprise any pharmaceutically acceptable carrier for the use to which the composition is put: notably a carrier for injection.

In addition to the bioactive glasses of the invention, it can be envisaged that formulations to be injected or put in place by surgery also comprise one or more compounds selected from antibiotics, antivirals, cicatrizing agents, antiinflammatories, immunosuppressants, growth factors, anticoagulants, vascularizing agents, analgesics, a plasmid, etc.

The materials of the invention can also be deposited on a metallic or ceramic element such as a screw, a plate, a tube, a valve etc., which is implanted in the organism as a prosthesis.

The materials of the invention can be combined with a matrix, such as a bone matrix that is intended to be grafted. Combination of the materials of the invention with the graft, notably when the latter is allogenic, promotes its incorporation in the organism.

Prostheses covered with a material of the invention can be manufactured in a known manner by immersing a conventional prosthesis, of metal or ceramic, or a bone graft after removal of its cellular network, or a biocompatible polymer, in a sol-gel solution or by plasma spraying of the composition on the prosthesis, then continuing with heating at a temperature above 600° C., which causes the bioactive glass to form.

A prosthesis, of metal or ceramic or polymer, or a bone matrix, covered partially or on their entire surface with a material of the invention, constitutes another object of the invention.

The materials of the invention can also be prepared by the sol-gel technique in the form of monoliths of controlled shape. According to this embodiment, the method comprises control of the stage of drying and densification so as to avoid cracks in the gel. Gelation of the sol is carried out at 60° C. in a container made of PTFE, the shape of which defines the final shape of the monolith.

These monoliths are used in surgery, for example for filling a bone defect.

The materials of the invention can also be introduced by surgery or by injection in a localization known for its brittleness of the bone, such as the neck of the femur in individuals with osteoporosis.

The materials of the invention can also be introduced around joints to promote repair and/or regeneration of the cartilage when it is damaged.

The materials and the compositions of the invention can be used for the repair of cartilage, either following injury that led to degradation of the cartilage, or within the scope of treatment of osteoarthrosis. Inflammatory diseases of the joints in general can constitute situations where the use of a material according to the invention can be beneficial.

The materials of the invention can also be prepared by a fusion process by mixing the metal oxides and the other components, heating them until fusion occurs, and then cooling them. The melting point is largely determined by the choice of components of the glass. It is between about 900 and 1500° C. The materials obtained in this case are monolithic and nonporous.

According to this embodiment, a glass frit can also be prepared, in a known manner, starting from the composition of molten glass, which is fritted to produce a particulate material.

In the case of materials obtained in the form of monoliths (fusion or sol-gel), these materials can be used in surgery, by insertion in a site to be treated, either because a bone defect needs to be filled or because salting out of strontium-treated apatite would be useful for reinforcing the osseous bone structure.

Another object of the invention comprises solutions obtained from bioactive glasses, by dissolving the bioactive glasses in an aqueous medium. These solutions can be produced by placing the bioactive glass of the invention in an aqueous solution, then leaving the glass to dissolve and filtering the medium. The filtered solution is recovered. It promotes the growth of osteoblasts. It can be used in a composition, notably an injectable composition, for administration in a localized region of the organism where it is desirable to stimulate the growth of osteoblasts. It can also be used in the laboratory for cell culture. It can be used for preparing a medicinal product of any form such as solid, semi-solid, liquid, for example in the form of tablets, pellets, powder, liquid solution, suspension, suppositories.

The materials, compositions and prostheses of the invention are particularly useful for repairing bone and/or cartilage defects and the deficiencies associated with diseases and injuries in the following cases: formation of bone tissues in a fracture, repair of bone defects such as those due to the ablation of a tumor or a cyst, treatment of dental or skeletal abnormalities, dental and periodontal reconstruction, notably replacement of alveolar bone, in periodontal diseases that lead to bone loss, or for filling a cavity between tooth and gum or for temporary replacement of an extracted tooth, in the case of osteoporosis.

A form is chosen that is suitable for the use to which it will be put, and most often a form permitting injection or surgical insertion at the site where an osseous deficiency has to be treated.

Another object of the invention consists of using a material as described above for the manufacture of a prosthesis or of a medicinal product intended for preventing or treating one or other of the pathologies described above.

The biomaterials that have been developed are nanostructured bioactive glasses. Physicochemical studies of the interactions between bioceramics and biological media reveal properties of bioactivity leading ultimately to the formation of a layer of calcium phosphate on the surface of the material. In the case of bioactive materials, this layer permits an intimate bond with the bone tissues. Moreover, by controlling the texture (porosity) and the content of principal elements and trace elements (Sr), it is possible to modulate the properties of dissolution and bioactivity of these materials. Thus, the glasses that have been developed salt-out strontium at physiological concentrations. This controlled salting out of a trace element (that is present in bone) can induce a specific response of the cells.

Various compositions of materials according to the present invention were produced and their behavior in solution was investigated. It was found that at the interface between the composition and the medium in which it is immersed, a hydroxyapatite forms, at a rate that can be controlled. It was also found that there is salting out of strontium in ionic form in the medium and its incorporation in the layer of calcium phosphate produced in situ.

Strontium, like calcium, is a network modifier in the compositions of the invention. Their ionic radii are similar. Nevertheless, it was found that the presence of strontium plays a role in the rate of salting out of the constituents of the composition, whereas calcium has little influence on this parameter.

It was found, in particular, that increase in the amount of strontium in the composition led to a decrease in the rate of salting out of strontium, calcium, phosphorus, and silicon.

Thus, the compositions of the invention not only permit salting out of strontium in their environment when they are placed in a physiological fluid, they also permit it to be done in a controlled manner.

To summarize, the compositions of the invention permit:
at physiological concentrations, salting out of strontium directly at the site of implantation,
improvement of bone mineralization,
control of dissolution and salting out of materials,
possibility of implanting and injecting the material at the chosen site,
formation of a layer of calcium phosphate at the periphery of the materials in a biological medium.

### EXPERIMENTAL SECTION

### I—Synthesis Protocol

The bioactive glasses were produced in the form of powders. The chemical precursors supplied by Sigma-Aldrich (USA) are presented in Table I-1.

TABLE I-1
Characteristics of the chemical precursors

| | | Molar | |
|---|---|---|---|
| | | mass | Purity |
| | Formula | (g · mol⁻¹) | (%) |
| Tetraethoxysilane | C₈H₂₀O₄Si | 208.33 | 99.999 |
| (TEOS) | | | |
| Triethylphosphate | C₆H₁₅O₄P | 182.16 | 99.8 |
| (TEP) | | | |
| Calcium nitrate | Ca(NO₃)₂—4H₂O | 236.15 | 99.99 |
| tetrahydrate | | | |
| Strontium | Sr(NO₃)₂ | 211.63 | 99 |
| nitrate | | | |

A cosolvent (ethanol EtOH) was used for carrying out the reaction of hydrolysis of the TEOS. Hydrochloric acid HCl was used as catalyst.

Regarding the synthesis protocol, the distilled water required for hydrolysis is first mixed with hydrochloric acid HCl (2N) and with ethanol EtOH (99%), which as well as giving a homogeneous solution after introduction of the TEOS, ensures good dissolution of the crystals of Ca(NO₃)₂-4H₂O. The proportions of water, of ethanol and of hydrochloric acid are detailed in Table I-2.

TABLE I-2
Proportions of water, ethanol and hydrochloric acid

| | |
|---|---|
| H₂O/(TEOS + TEP) | Rₘₒₗₐᵣ = 12 |
| H₂O/HCl | R_{volumetric} = 6 |
| H₂O/EtOH | R_{volumetric} = 1 |

These reactants are mixed in a flask with magnetic stirring for 15 minutes. The TEOS is then added to the mixture and, after 30 minutes, the TEP is poured in together with an equal volume of ethanol. After 20 minutes, crystals of Ca(NO₃)₂-4H₂O are introduced. The mixture is then stirred for a further 60 minutes.

Then the solution is placed in a watch glass and dried in a stove at 60° C. for gelation. This operation takes 4 hours, and leads to complete gelation of the sol. The stove temperature is then raised to 125° C. for 24 hours. The gel is now completely fragmented and it is ground finely in a mortar for the last stage of synthesis: calcination at 700° C. for 24 hours, which in addition to densification will ensure complete evaporation of the residues of alcohol and of nitrate trapped in the pores. The final product is obtained in the form of a fine white powder.

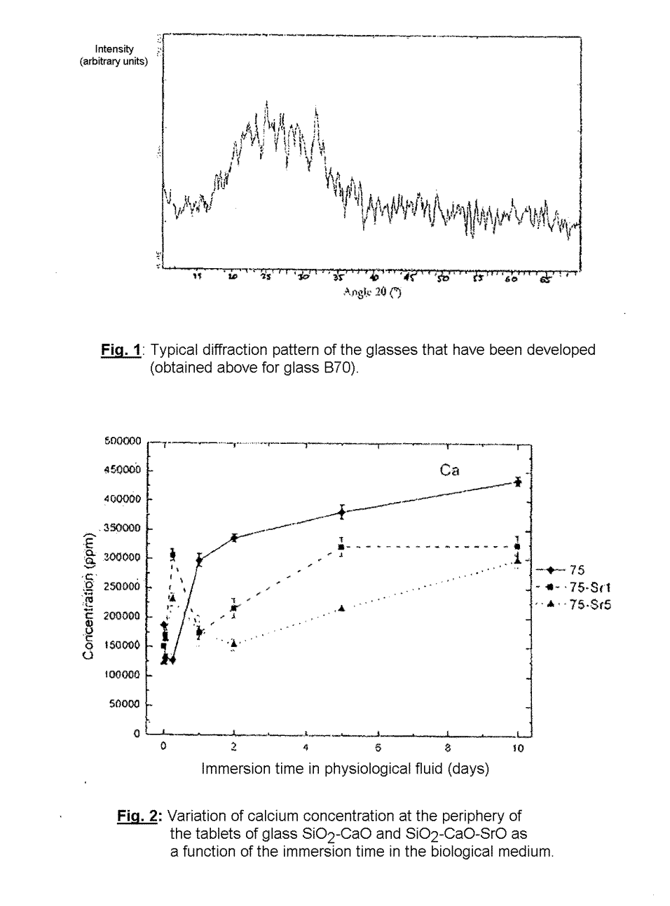

FIG. 1 shows the diffraction pattern for a glass, characterized by X-ray diffraction crystal analysis. The diffraction patterns obtained for the other glasses are similar to this one. The absence of diffraction peaks indicates that the glasses that have been developed are indeed amorphous.

### II—Characteristics of the Glasses Developed

II-1—Composition

The composition of the glasses developed was investigated by atomic emission spectrometry (ICP-AES). The results of analysis by atomic emission spectrometry are presented in Tables II-1 and II-2. The glasses developed have concentrations of oxides according to the expected values.

TABLE II-1
Concentrations of oxides measured by ICP-AES
for the binary and ternary glasses (wt. %).

| | B75 | B72.5 | B70 | B67.5 |
|---|---|---|---|---|
| SiO₂ Theoretical | 75 | 72.5 | 70 | 67.50 |
| SiO₂ Experimental | 72.20 | 71.49 | 68.25 | 63.75 |
| P₂O₅ Theoretical | — | 2.5 | 5 | 7.5 |
| P₂O₅ Experimental | — | 2.48 | 4.85 | 6.95 |
| CaO Theoretical | 25 | 25 | 25 | 25 |
| CaO Experimental | 24.50 | 24.36 | 25.76 | 24.13 |

TABLE II-2
Concentrations of oxides measured by ICP-AES
for the strontium-doped glasses (wt. %).

| | B75-Sr1 | B75-Sr5 | B67.5-Sr1 | B67.5-Sr5 |
|---|---|---|---|---|
| SiO₂ Theoretical | 75 | 75 | 67.5 | 67.5 |
| SiO₂ Experimental | 74.22 | 74.08 | 67.16 | 64.93 |
| P₂O₅ Theoretical | — | — | 7.5 | 7.5 |
| P₂O₅ Experimental | — | — | 7.04 | 7.62 |
| CaO Theoretical | 24 | 20 | 24 | 20 |
| CaO Experimental | 23.60 | 19.03 | 23.31 | 20.25 |
| SrO Theoretical | 1 | 5 | 1 | 5 |
| SrO Experimental | 0.83 | 3.83 | 0.81 | 4.27 |

II-2—Texture

The specific surfaces of the glasses were measured by gas adsorption on an Autosorb Quantachrome instrument operating at 77.4 K by the BET method. The adsorbate used is high-purity nitrogen (99.999%) with an effective adsorption cross-section of the nitrogen molecule of 0.162 nm² for calculation of the specific surface. Prior to measurement, the samples are degassed under vacuum (p<1 Pa) at 120° C. for 12 h. The specific surfaces are calculated from the masses of the degassed samples.

At least 5 points were used for measuring the amounts of gas adsorbed in a range of partial pressure p/p₀ between 0.05 and 0.3 (with P₀: saturated vapor pressure).

The specific surfaces are between 50 and 150 m²/g. The average pore size is between 1 nm and 101 nm.

### III—Investigation of Bioactivity In Vitro

It has been clearly established that the ability of a biomaterial to bind to living tissues is dependent on its capacity for forming a layer of apatite in contact with biological fluids simulating blood plasma. Tests in vitro are therefore a powerful tool for evaluating the bioactivity of a material.

The biological medium in which the bioactive glasses were immersed is DMEM (Dulbecco's Modified Eagle Medium). The composition of DMEM is similar to that of human blood plasma (Table III-1). The pH of DMEM at 37° C. is 7.43, a value similar to that of plasma.

TABLE III-l
Ionic concentrations of human blood plasma (mmol · L⁻¹).

| Na⁺ | K⁺ | Mg²⁺ | Ca²⁺ | Cl⁻ | CO₃²⁻ | PO₄³⁻ | SO₄²⁻ |
|---|---|---|---|---|---|---|---|
| 142.0 | 5.0 | 1.5 | 2.5 | 147.8 | 4.2 | 1.0 | 0.5 |

III-1—Experimental Protocol

The samples of bioactive glasses were investigated in the form of powder and in the form of tablets: disks 13 mm in diameter and with a height of 2 mm, obtained by compacting 90 mg of powder in a press. The bioactive glasses can be used in clinical applications in these two forms, and it is therefore interesting to investigate these two types of samples. The bioactivity operates on different scales of time and dimensions.

III-1.1—Samples in the Form of Tablets

The tablet samples were immersed in 45 mL of DMEM for the following times: 1 hour, 6 hours, 1 day, 2 days, 5 days, 10 days.

After immersion, the tablets are recovered and then dried in ambient atmosphere, whereas a sample is taken from the DMEM for analysis by ICP-AES. The samples of tablets intended to be characterized using the ionic microprobe are embedded in resin. Cross-sections of the material are then prepared using a Leica RM 2145 microtome. The sections, 30 μm thick, are cut perpendicularly to the surface of the disk. Finally, the sections are placed on Mylar supports pierced centrally with a hole with a diameter of 3 mm. It is the region of the sample located above the hole that is probed by the ion microbeam.

III-1.2—Samples in the Form of Powder

Not being massive like the tablet samples and moreover having a porous structure, the samples of powder grains react more quickly than the tablet samples. Our investigation of the powders focused on the characterization of 4 bioactive glasses: glasses B75, B67.5, B75-Sr5 and B67.5-Sr5. For each glass, 10 mg of powder was immersed in DMEM according to a ratio [specific surface]/[volume of DMEM] fixed at 500 cm⁻¹, in order to evaluate the effect of the composition of the glass, rather than its area of contact with the biological medium, on the bioactivity. The following immersion times in DMEM were used: 1 hour, 6 hours, 1 day, 2 days, 3 days, 4 days.

III-2—Characterization of Physicochemical Reactions During Interactions Between the Bioactive Glass and the Biological Medium

For a better understanding of the physicochemical reactions leading to the formation of different layers at the periphery of the bioglasses, it is essential to undertake a local analysis of the distribution of the elements at the interface between the materials and biological fluids. These analyses require the use of methodologies having good sensitivity and excellent spatial resolution. For this purpose we carried out chemical cartography of the interface at the micrometer scale by the PIXE method (Particle Induced X-ray Emission). This method is based on X-ray fluorescence induced by a beam of ions (generally protons). It can be used for simultaneous multi-element cartography and measurements of concentrations for the major, minor and trace (ppm) elements with a spatial resolution of the order of a micron.

III-2.1—Multi-Element Chemical Imaging at the Periphery of Tablets of Strontium-Doped Glass after Immersion in the Biological Medium

The multi-element cartographs were recorded during the ion microbeam analysis of tablets of strontium-doped binary and ternary glasses. Multi-element cartographs were obtained for each of the glasses before interaction and after 1 hour, 6 hours, 1 day, 2 days, 5 days and 10 days of interaction with the biological medium. The discussion draws comparisons with the investigation of tablets of non-doped binary and ternary glasses.

Based on the chemical images obtained, it was possible to monitor the distribution of silicon, calcium, phosphorus, strontium and magnesium at the bioactive glass/biological medium interface as a function of the time of interaction between the material and the liquid. Measurements of concentrations in the glasses carried out by PIXE then supply local information on the reactivity of the material. In order to obtain information on the overall reactivity of the samples, the variation of the concentrations in the biological medium was monitored with measurements by ICP-AES. Comparison of these results is therefore necessary, and will provide additional information on the reactivity of the material. Regarding the PIXE analysis, the tablet samples were probed with a proton beam with diameter of 2 μm and intensity of 500 pA. The cartographs were obtained by scanning square regions with side between 40 μm and 400 μm depending on the regions of interest.

The cartographs of the glasses of composition SiO₂—CaO—SrO reveal that addition of strontium to the composition of the glass reduces the dissolution of the material compared with an SiO₂—CaO glass. This effect can be seen in the cartographs for calcium.

Regarding the distribution of strontium, this element is distributed uniformly up to 1 hour of interaction. Some of the strontium then appears to be salted-out from the periphery of the material, and strontium is detected in higher proportions in the interior regions of the tablets.

Doping with strontium is also found to have an effect on the development of the layer of calcium phosphate. Thus, whereas the presence of phosphorus was detected at the periphery of glass B75 after just 1 hour of interaction, this element is only detected after 6 hours of interaction for glasses B75-Sr1 and B75-Sr5. Moreover, traces of magnesium are only detected after 6 hours for glasses SiO₂—CaO—SrO, in contrast to 1 hour for SiO₂—CaO glass. Thereafter, the Ca—P—Mg layer grows in a similar manner to the binary glass. After 10 days of interaction, three regions are observed in these glass tablets. The innermost regions of the tablet are constituted of the original vitreous network. The peripheral layer is an extensive region rich in calcium and phosphorus, where there are traces of magnesium and strontium. Finally, between these two regions, we find an intermediate region with local calcium enrichment.

The multi-element cartographs for the glasses of composition SiO₂—CaO—P₂O₅—SrO also indicate a slowing of the dissolution of the material in comparison with the undoped glass B67.5. Addition of strontium is thus reflected in slowing of the salting out of calcium. The ability of these materials to form a Ca—P—Mg—Sr layer is nevertheless demonstrated after some days of interaction.

III-2.2—Local Measurements of the Concentrations of Elements During Interactions Between Glass Tablets and Biological Medium

Depending on the distribution of the chemical species, the multi-element cartography were divided up into various regions of interest. Whenever the peripheral region rich in calcium and phosphorus was identified, measuring masks were defined, enabling the concentrations of elements there to be calculated. Depending on the region of interest, masks with a side from 5 to 20 μm were defined, and the thickness of the Ca—P layer increased with the immersion time. Applying this methodology, it was possible to monitor the evolution of the concentrations of the species Ca, P, Si, Sr and Mg at the periphery and at the center of the glasses. For a given time of interaction and a given glass, the concentration values shown on the graphs are the mean concentrations found in several regions of interest.

Evolution of the Concentrations at the Periphery of the Glass Tablets

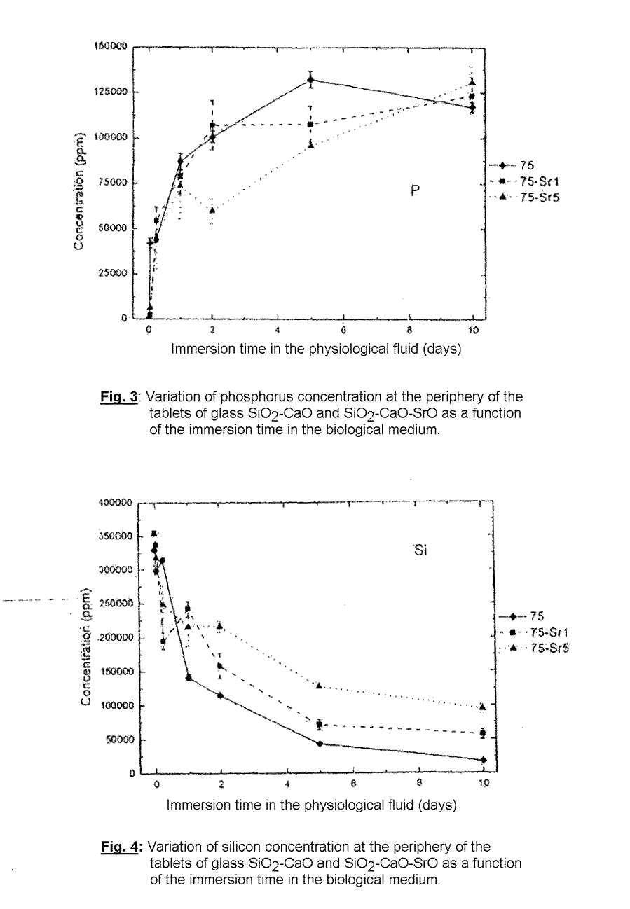

FIGS. 2, 3, 4 and 5 show the evolution of the concentrations of elements at the periphery of the SiO₂—CaO—SrO glasses. The concentrations measured at the periphery of glass B75 (SiO₂—CaO) are also shown, for comparison. FIG. 2, presenting the evolution of the calcium concentrations, shows that glasses B75-Sr1 and B75-Sr5 display a behavior substantially different from that of glass B75. For the SiO₂—CaO—SrO glasses, the calcium concentration begins to increase during the first few hours of interaction with the physiological fluid. The relative increase in calcium concentration is due to the fact that at the same time there is a sharp drop in the silicon concentration (FIG. 4). This tends to indicate that in the strontium-doped glasses, salting out of calcium does not progress as quickly as the decomposition of the silicon network: the salting out of calcium is slowed down and seems to affect a more limited number of cations of the matrix. It is not until after 6 hours of interaction that the calcium concentration drops to a minimum, reached after 1 day of interaction for glass B75-Sr1 and after 2 days for glass B75-Sr5. The minimum reached is higher than for glass B75: dissolution is therefore less complete for the materials doped with strontium.

After the salting-out stage, the amount of calcium present at the periphery of the glasses increases, but this increase is less rapid for the SiO₂—CaO—SrO glasses than for the SiO₂—CaO glass. After immersion for 10 days, the proportion of calcium contained in the peripheral Ca—P layer of the strontium-doped glasses is close to 30 wt. %, which is less than the amount of calcium detected in the peripheral Ca—P layer of glass B75 (44 wt. %). It must be borne in mind, however, that the matrixes of glasses B75-Sr1 and B75-Sr5 contain less calcium initially.

FIG. 4 shows that the decrease in silicon concentration at the periphery of the material is slower with increasing proportion of strontium in the original vitreous matrix. After 10 days of interaction, the peripheral layer of glass B75-Sr1 is still composed of 6% silicon, and that of glass B75-Sr5, 9% silicon.

Concerning phosphorus (FIG. 3), there appears to be a common trend for the three glasses B75, B75-Sr1 and B75-Sr5; namely, a rapid increase in concentration of this element at the periphery of the tablets. An extremum is finally reached after 10 days of interaction. The phosphorus content of the periphery of glasses B75, B75-Sr1 and B75-Sr5 is then close to 12%.

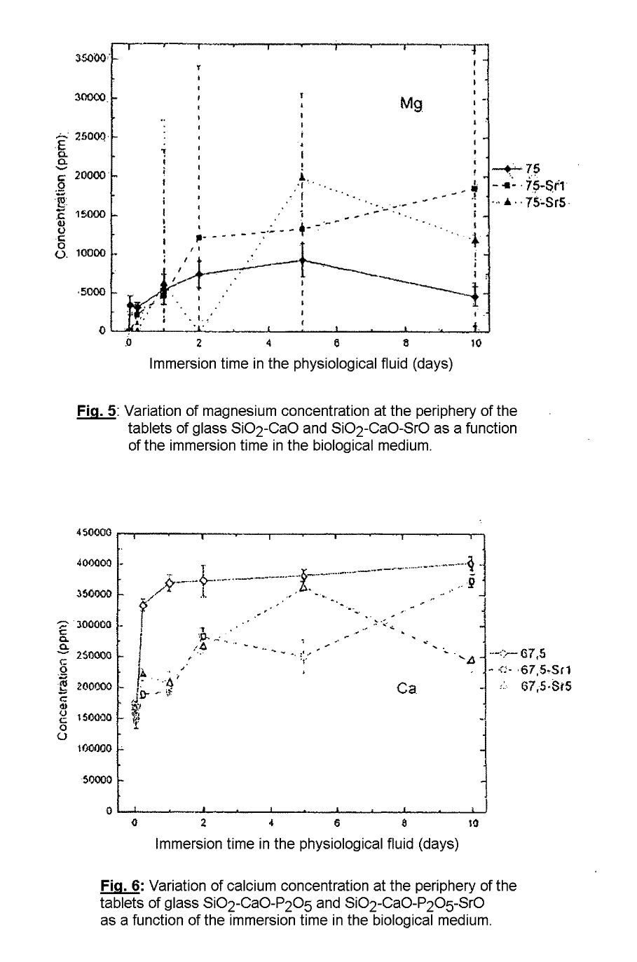

Traces of magnesium are detected in the layer that develops on the surface of the SiO₂—CaO—SrO glasses (FIG. 5). The proportion of magnesium increases with longer immersion time and therefore as the peripheral layer extends to the surface of the glasses. The amount of magnesium incorporated at the periphery of the tablets is found to be greater for the SiO₂—CaO—SrO glasses than for the SiO₂—CaO binary glass.

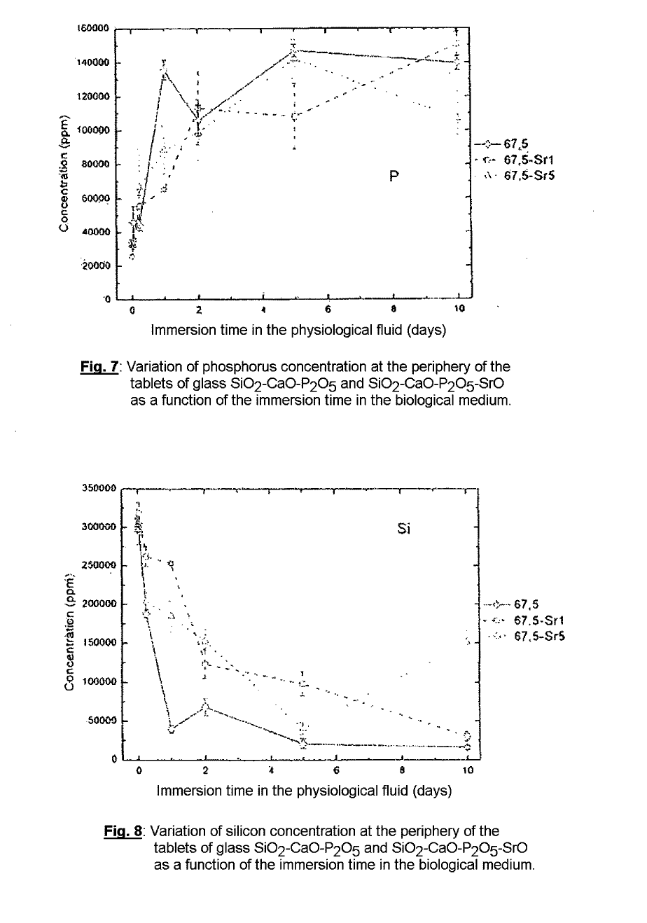

FIGS. 6, 7, 8 and 9 show the evolution of the concentrations of elements at the periphery of the SiO₂—CaO—P₂O₅—SrO glasses. It can be seen in FIG. 6 that the evolution of the calcium concentrations at the periphery of glasses B67.5-Sr1 and B67.5-Sr5 increases similarly to that of glass B67.5; however, the variation is slower and calcium is present in smaller amount for the strontium-doped glasses. It can be seen from FIG. 8 that the kinetics of decrease in silicon concentration is less rapid for the glasses containing strontium than for the ternary glass B67.5. After 10 days of interaction, moreover, there are still high concentrations of silicon at the periphery of the SiO₂—CaO—P₂O₅—SrO glasses. These observations are an indication that in the strontium-doped glasses, decomposition of the vitreous network goes to a smaller depth.

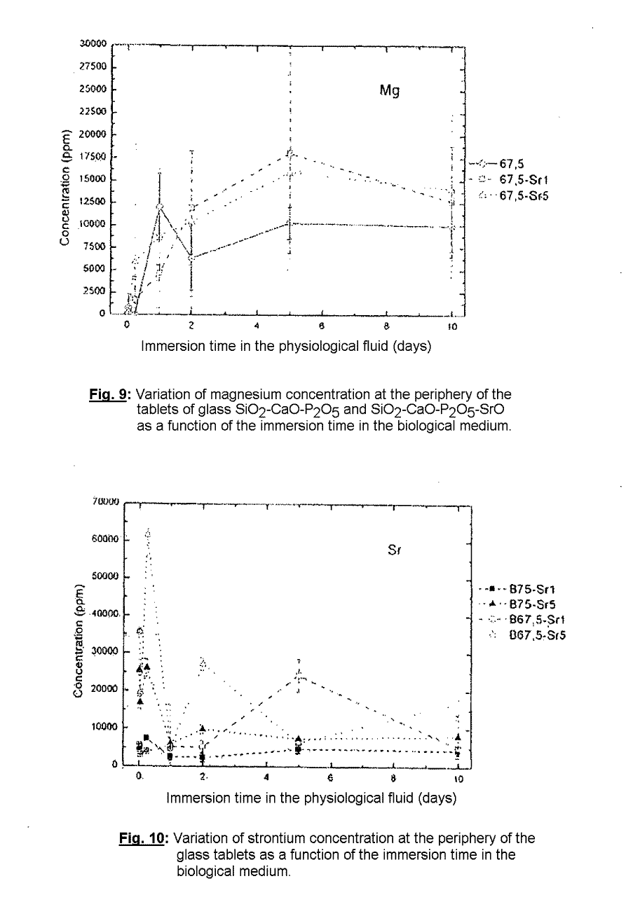

The amount of phosphorus detected in the peripheral region of the tablets increases rapidly with the immersion time (FIG. 7). The variation in concentrations is common to the three glasses and the peripheral layer is at the end constituted of 11 to 15% phosphorus. Regarding magnesium, after 10 days this element is present at a level of 1% at the periphery of the tablets. It can be seen from FIG. 9 that a larger amount of magnesium is incorporated in the glasses composed of strontium B67.5-Sr1 and B67.5-Sr5 compared with glass B67.5.

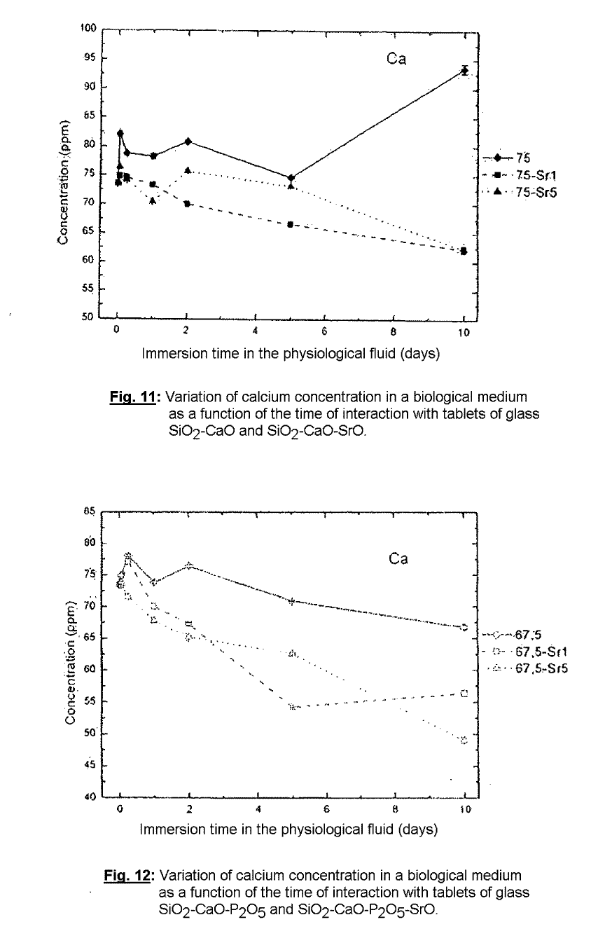

FIG. 10 shows the variation in concentrations of strontium at the periphery of the tablets of SiO₂—CaO—SrO and SiO₂—CaO—P₂O₅—SrO glass. Under the influence of ion exchanges and the physicochemical reactions taking place at the surface, large fluctuations in the concentration of strontium are observed. Nevertheless, for the peripheral layer it can be seen that there is a general tendency for slight depletion of strontium. After 10 days of interaction, the periphery of the materials is richer in strontium with higher proportion of this element in the original vitreous matrix, and the measured concentrations are lower than the values before interaction.

Variation of Concentrations in the Interior Region of the Glass Tablets

Measurements of the concentrations of elements in the interior regions of the glass tablets, not directly exposed to the biological fluids, were effected for the elements Si, Ca, P, Sr and Mg. As noted previously, the phenomena of diffusion and of migration of ions toward the periphery of the material lead to fluctuations in the composition of the vitreous matrix. The principal variations are observed for the concentrations of silicon, calcium and strontium during the first 2 days of interaction with the biological medium. The variation in the concentration of phosphorus also shows a slight tendency to increase. After 10 days of interaction, the concentrations of the various constituent elements are observed to return to a value close to their value in the original vitreous matrix. The interior regions of the tablets of strontium-doped glasses have changed less than the undoped glasses. The amplitude and the kinetics of dissolution are lower in the doped glasses, and the Ca—P—Mg layer that developed at the periphery does not appear to extend to the innermost regions of the glass tablets.

Variation of the Atomic Ratios at the Interface Between the Glass Tablets and the Biological Medium

The variation of the atomic ratios Ca/P, Ca/Mg and Ca/Sr at the interface between the glass and the biological medium was investigated.

During the first few hours of interaction, the atomic ratio Ca/P is higher for the SiO₂—CaO—SrO glasses than for the SiO₂—CaO glass. This relates to the fact that calcium, salted-out in smaller amounts for the SiO₂—CaO—SrO glasses, is therefore present at higher proportions on the surface of these materials. Beyond 6 hours of interaction, the dissolution and salting out of calcium accelerate for glasses B75-Sr1 and B75-Sr5 and, combined with the rapid incorporation of phosphorus from the medium, this results in the sharp drop in Ca/P ratio observed at 1 day of interaction. Then, as the immersion time in the biological fluid increases, the Ca/P ratio tends to a limit value close to 1.7, which is that of the stoichiometric hydroxyapatite. Thus, after 10 days of immersion, it is found that the value of the Ca/P ratio finally reached is equal to 1.8 for glasses B75-Sr1 and B75-Sr5, which is closer to the nominal value of the stoichiometric apatite when compared with the result of 2.1 obtained for glass B75. Regarding the SiO₂—CaO—P₂O₅—SrO glasses: the Ca/P ratios measured at the interface are always lower than those of the SiO₂—CaO—P₂O₅ glass. This is due on the one hand to the smaller increase in calcium concentration, and on the other hand to the lower proportion of calcium initially present in these materials, respectively 24% and 20% for glasses B67.5-Sr1 and B67.5-Sr5, against 25% for B67.5. After 10 days of interaction, the comment made regarding the SiO₂—CaO—SrO glasses is also valid for the SiO₂—CaO—P₂O₅—SrO glasses, namely that the Ca/P ratio for the strontium-doped glasses is closer to that of the stoichiometric hydroxyapatite compared with the undoped glasses. After 10 days of immersion, this Ca/P ratio is 1.6 for glass B67.5-Sr1 and 1.7 for glass B67.5-Sr5, against 1.9 for glass B67.5.

Variation in the Composition of the Biological Medium During the Interactions with the Glass Tablets

The variation in concentration of calcium present in the DMEM (FIGS. 11 and 12) is slight during the first few hours of interaction. The amount of calcium salted-out in the medium during the surface dealkalization phase is lower for the strontium-doped glasses than for glasses B75 and B67.5. Then, as the immersion time increases, a gradual decrease in calcium concentration in the biological medium is found for all the doped glasses. The binary glass B75 salts out large amounts of calcium, so that this element was present at higher concentration after 10 days of interaction than before interaction; this observation is not found for glasses B75-Sr1 and B75-Sr5. The strontium-doped glasses are found to incorporate larger amounts of calcium compared with those salted-out. Thus, after 10 days of interaction, the calcium concentration in the biological medium is only 62 ppm for glasses B75-Sr1 and B75-Sr5, whereas it was 94 ppm for glass B75. For glasses B67.5-Sr1 and B67.5-Sr5, it is equal respectively to 5 and 49 ppm, whereas it was 67 ppm for glass B67.5.

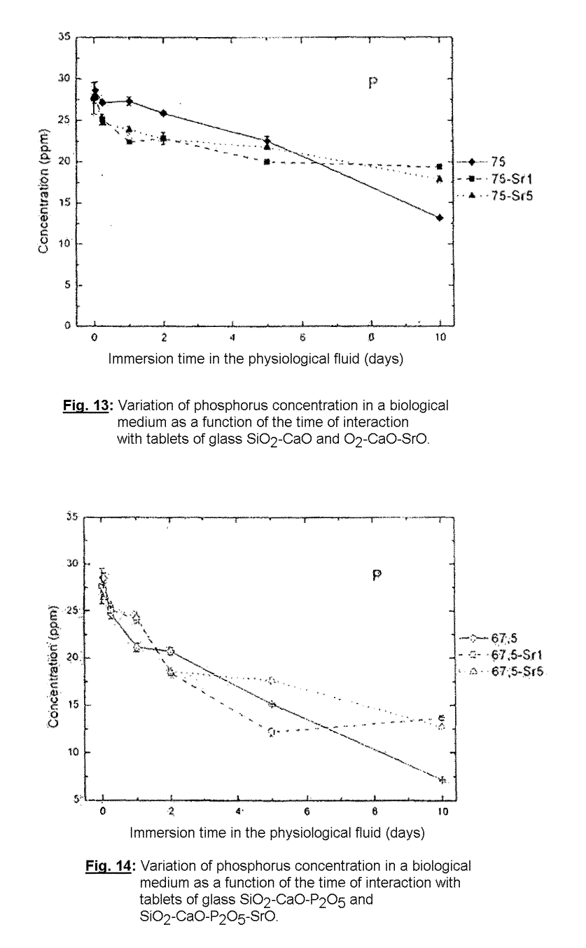

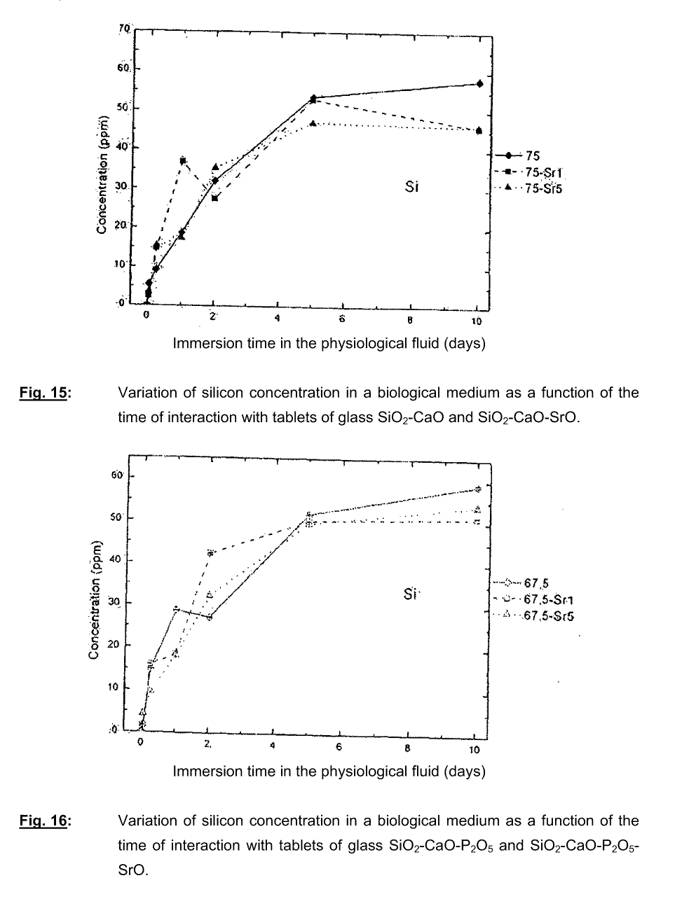

FIGS. 13 and 14 show the variation in the concentration of phosphorus present in the biological medium. For all the glasses, there is a large decrease in the concentration of this element over time. The decreases observed for each of the samples are similar. However, it can be seen that after 5 days of interaction that the consumption of phosphorus appears to have slowed in the strontium-doped glasses. This might be an indication that the glass-biological medium system is approaching equilibrium.

Regarding silicon, FIGS. 15 and 16 show a common trend for all the glasses. As the reactions of dissolution break down the vitreous network to ever increasing depths, higher and higher concentrations of silicon are detected in the biological medium. After 10 days of interaction, the amounts of silicon salted-out in the biological medium are lower for the strontium-doped glasses. This is another indicator of the lower degree of dissolution in the doped glasses.

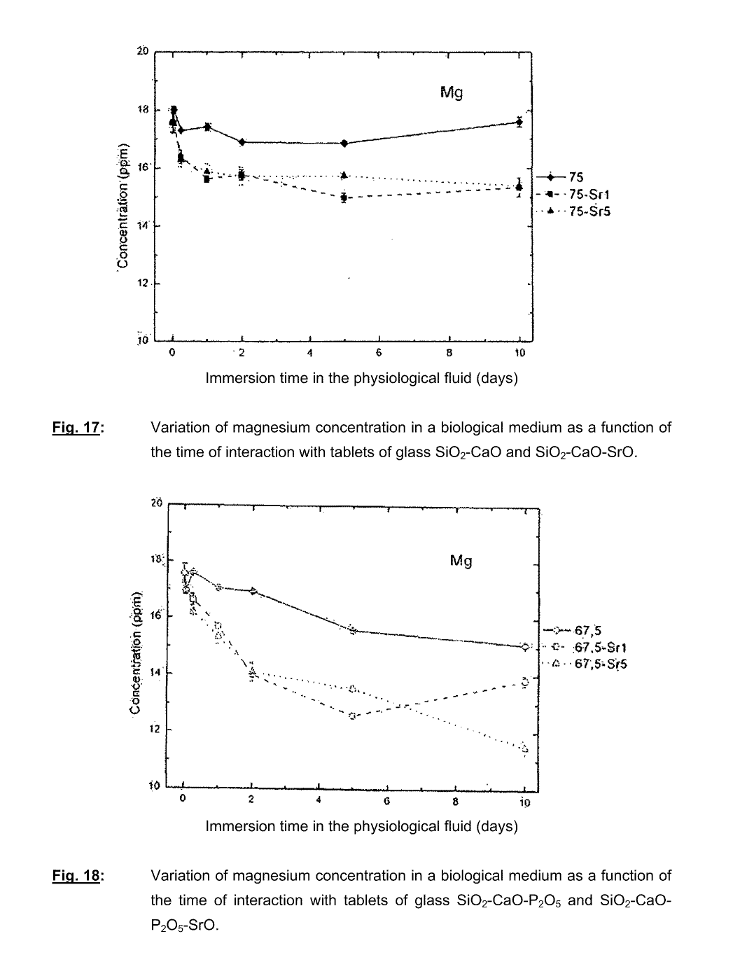

On the other hand, FIGS. 17 and 18 show that the strontium-doped glasses incorporate more magnesium than the other glasses. The concentration of this element decreases slowly with the immersion time, and after 10 days the decrease in magnesium is 2 ppm for glasses B75-Sr1 and B75-Sr5, 3 ppm for glass B675-Sr1 and 5 ppm for glass B67.5-Sr5.

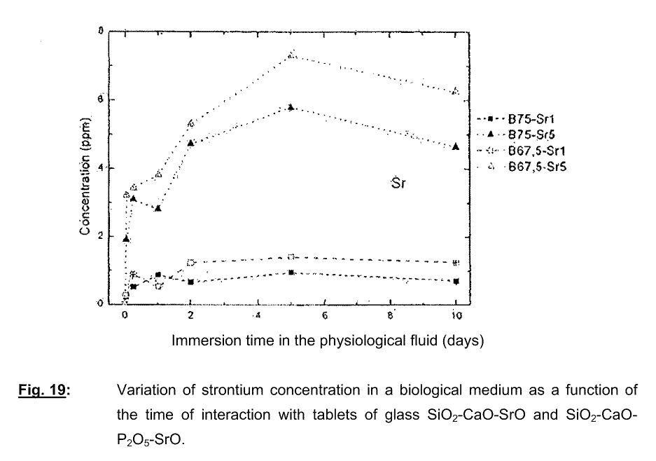

Finally, measurements of the concentration of strontium present in the biological medium complete this study (FIG. 19). Initially equal to zero, the amount of strontium in the physiological fluid increases to a few ppm after salting out of this element away from the surface of the glasses. It can be seen that glasses B75-Sr5 and B67.5-Sr5 salt out 5 times more strontium than glasses B75-Sr1 and B67.5-Sr1, which tallies with the respective strontium contents of these materials.

III-2.4—Local Measurements of the Concentrations of Elements During Interactions Between Glass Grains and the Biological Medium

Variation of the Concentrations at the Periphery of the Glass Grains

Local analysis of the periphery of the grains reveals that the phenomena observed for powders reproduce those observed for the tablets, but at reduced scales of time and dimensions. The concentrations of elements display trends similar to those observed previously.

These observations also apply to the variation in the concentration of phosphorus. Just as for the tablets, the concentration of this element increases rapidly at the periphery of the grains. After 4 days of interaction, phosphorus is contained there at a level of 9-10% for the strontium-doped glasses, and at a level of 16% for the ternary glass. For glass B75, the amount of phosphorus increases rapidly until 6 hours of interaction. After that, the phosphorus concentration decreases almost to zero. The layer of calcium phosphate formed at the boundary of the grains in glass B75 therefore appears to be unstable and it is quickly dissolved under the action of biological fluids.

It is found that the silicon concentration at the grain boundaries in glass B75 decreases in the early stages of interaction, corresponding to breakdown of the vitreous network at the periphery of the material. However, beyond 6 hours of interaction, the concentric layer of calcium phosphate is dissolved and consequently the grains now only comprise a silicon-rich vitreous core. For glasses B67.5, B75-Sr5 and B67.5-Sr5, a different phenomenon is observed: the silicon network is gradually broken down in the peripheral regions of the grains, and as a result the concentration of this element decreases. It will be noted that the decrease is slower for the strontium-doped glasses compared with the undoped glasses; this was also the case for the samples in the form of tablets.

### IV—Preliminary Evaluation of the Behavior of Osteoblasts Cultivated in Contact with Strontium-Doped Bioglass (B75Sr5)

IV-I—Investigation in vitro—Method of culture

The strontium-doped bioglasses B75Sr5 are investigated in the form of granules. Prior to use, the bioglasses are weighed and sterilized at 180° C. for 2 hours. The granules are then pre-incubated in the culture medium (see composition below) for 48 hours, with stirring. Following this preincubation, the granules of bioglasses are put in contact with the cells immediately.

Osteoblasts are isolated by enzymatic digestion from calvaria of rat fetuses aged 21 days. The calvaria are dissected in sterile conditions and the fragments are incubated in the presence of collagenase (Life Technologies®) for 2 hours at 37° C. The cells dissociated from the bone fragments are then seeded in culture dishes (5 ml) at a density of 2.10⁵ cells/ml. When the culture reaches the stage of subconfluence (about 80% of the surface colonized), the granules of bioglasses are added to the lawn (20 mg/culture dish). The culture medium is composed of DMEM (Invitrogen®), ascorbic acid (50 μg/mL), 10 mM of β-glycerophosphate (Sigma®), 50 IU/mL of Penicillin-Streptomycin (Gibco®) and 10% of fetal calf serum (FCS) (Hyclone®). The cells are cultivated for 14 days, in an incubator at 37° C. in a humid atmosphere at 5% CO₂.

IV-2—Investigation of the Interface Between the Grain of Glass B75Sr5 and Bone Cells by Phase-Contrast Photonic microscopy

Observations by phase-contrast photonic microscopy make it possible to follow the development, maturation and the formation of bone nodules around and in contact with the bioglass.

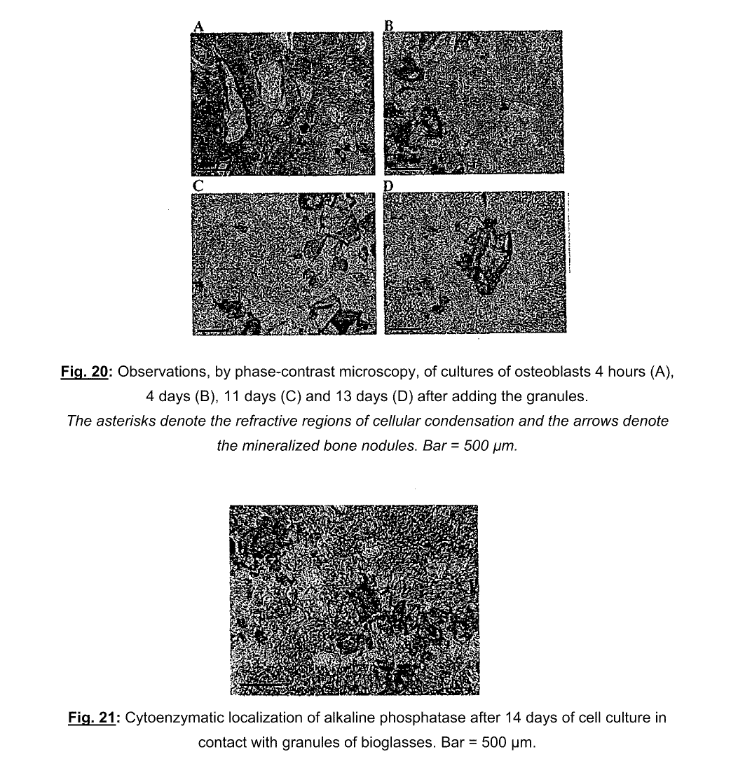

During the first few days of culture, the cells proliferate (FIG. 20) and reach confluence between the 3rd and 4th day of culture (FIG. 20), immobilizing the granules in the lawn. During the days that follow, the cells continue to proliferate and become arranged in multilayer films at the periphery of the granules. This three-dimensional arrangement can be seen from the start of the second week of culture in the form of refractive regions (FIG. 20). At the end of the second week of culture, these refractive regions are very abundant around the granules and begin to spread onto the whole lawn, and starting from the 13th day we observe the appearance of the first mineralized bone nodules (FIG. 20).

These results demonstrate that in the presence of granules of strontium-doped bioglasses, the rat calvarial cells proliferate and differentiate into active osteoblasts, which form mineralized bone nodules.

IV-3—Cytoenzymatic Localization of Alkaline Phosphatase

The cells are cultivated for 14 days in contact with the bioglass granules. These cells are then fixed in a fixing solution (mixture of citrate and acetone) at room temperature for 30 seconds. The cellular samples are then rinsed and incubated in a solution that stains the cells synthesizing alkaline phosphatase (solution of “fast blue salt RR” and naphthol phosphate, Sigma®) at room temperature for 30 minutes, protected from the light. After the cytoenzymatic reaction, the samples are rinsed and then are examined by phase-contrast photonic microscopy.

On the 14th day of culture, positive labeling of alkaline phosphatase, a marker of osteoblast differentiation, is observed for the cells located around and in contact with the granules of bioglasses (FIG. 21). These results indicate that the presence of bioglasses of type B75Sr5 permits differentiation of rat calvarial cells.

IV-5—Investigation by Light Microscopy and Transmission Electron Microscopy

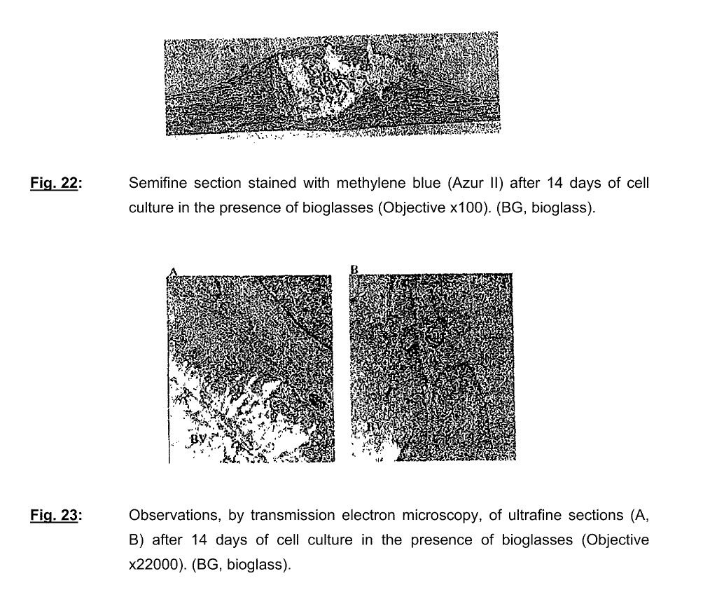

The cells are treated for transmission electron microscopy after 14 days of culture in contact with the granules of bioglasses. The cells are fixed in Karnovsky solution (4% paraformaldehyde and 1% glutaraldehyde) and then the samples are dehydrated with increasing ethanol baths. The lawn with the immobilized granules is then embedded in Epon-Araldite, and semifine sections (FIG. 22) and then ultrafine sections (FIG. 23) are prepared with a diamond cutter, perpendicular to the lawn. The ultrafine sections are collected on copper grilles and are then stained with uranyl acetate and lead citrate. The sections are then examined with a transmission electron microscope (Philips CM-12).

Three-dimensional arrangement of multilayer films of the cells around the granules is observed on the semifine sections (FIG. 22).

The observations in transmission electron microscopy reveal the presence of numerous cells in contact with the granules (FIG. 23). These cells have developed intracytoplasmic organelles, indicating vigorous cellular activity. They are surrounded by a dense extracellular matrix rich in collagen fibers. We can also observe multiple foci of mineralization in the matrix. Finally, intimate contact is observed between the matrix, the cells and the periphery of the granules.

The presence of the bioglasses does not alter the capacities for matrix synthesis, since the cells display all the signs of synthetic activity (endoplasmic reticulum, mitochondria, large nucleus, etc.). We also observe the presence of an extracellular matrix composed of numerous collagen fibers.

Conclusions Relating to the Biological Study

These results, taken together, demonstrate the noncytotoxic character of the granules of strontium-doped bioglasses on the primary cells obtained from rat calvaria. In fact, after 14 days of culture in contact with the bioglasses, no sign of cellular distress is detected and the cells cultivated in contact with these granules proliferate, organize into a three-dimensional structure and are capable of synthesizing an extracellular matrix. Moreover, the alkaline phosphatase activity of these cells and the appearance of mineralized bone nodules after 2 weeks of culture indicate that the presence of the granules of bioglasses is not harmful to the osteoblast differentiation of the cells investigated.

1. A material, characterized in that it results from a sol-gel process and that its composition is characterized by the presence of the following elements in the proportions stated:
SiO₂: from 40 to 75%
CaO: from 15 to 30%
SrO: from 0.1 to 10%
P₂O₅: from 0 to 10%
Na₂O: from 0 to 20%
MgO: from 0 to 10%
ZnO: from 0 to 10%
CaF₂: from 0 to 5%
B₂O₃: from 0 to 10%
Ag₂O: from 0 to 10%
Al₂O₃: from 0 to 3%
MnO: from 0 to 10%
Others: from 0 to 10%
the percentages being percentages by weight relative to the total weight of the material.

2. The material as claimed in claim 1, characterized in that the sum of the weights of the constituents SiO₂, CaO, SrO, P₂O₅ represents 98 to 100% of the total weight of the composition of the materials of the invention.

3. The material as claimed in claim 1 or 2, characterized in that its composition is as follows:
SiO₂: from 45 to 75%
CaO: from 15 to 30%
SrO: from 2 to 8%
P₂O₅: from 0 to 10%
Other elements: from 0 to 1%
by weight relative to the total weight of the composition.

4. The material as claimed in claim 1, characterized in that it is in the form of a loose powder or a compacted powder, in the form of fibers, in the form of a monolith or in the form of a glass frit.

5. The material as claimed in claim 1, characterized in that it is in the form of a coating on a substrate.

6. The material as claimed in claim 4, characterized in that it is in the form of powder and that it has pores with a size between 1 nm and 50 μm.

7. A material, characterized in that it results from a fusion process and that its composition is characterized by the presence of the following elements in the proportions stated:
SiO₂: from 45 to 55%
Na₂O: from 10 to 25%
CaO: from 10 to 25%
SrO: from 0.1 to 10%
P₂O₅: from 0 to 10%
MgO: from 0 to 10%
ZnO: from 0 to 10%
CaF₂: from 0 to 5%
B₂O₃: from 0 to 10%
Ag₂O: from 0 to 10%
Al₂O₃: from 0 to 3%
MnO: from 0 to 10%
Others: from 0 to 10%
the percentages being percentages by weight relative to the total weight of the material.

8. The material as claimed in claim 7, characterized in that the sum of the constituents SiO₂, Na₂O, CaO, SrO, P₂O₅ represents 98 to 100% of the total weight of the materials of the invention.

9. The material as claimed in claim 8, characterized in that it is in the form of a monolith or a glass frit.

10. A therapeutic composition, comprising the material as claimed in claim 1 and a pharmaceutically acceptable carrier.

11. A bone prosthesis or matrix with its surface covered partially or completely with a material as claimed in claim 1.

12. A solution obtained from a material as claimed in claim 1, by dissolving the material in an aqueous medium.

13. A method for the filling of a bone defect comprising administering to a bone defect the material as claimed in claim 1.

14. A method for the stimulation of bone growth comprising administering to a bone the material as claimed in claim 1.

15. A method to promote repair and/or regeneration of cartilage comprising administering to cartilage the material as claimed in claim 1.
